(19) United States
(12) Patent Application Publication
(10) Pub. No.: US 2019/0231537 A1
(43) Pub. Date: Aug. 1, 2019

(54) METHOD FOR THE PRODUCTION OF A MANDIBULAR JOINT ENDOPROSTHESIS OR OF AN IMPLANT FOR TREATING BONE DEFICIENCIES OR DEFECTS OF THE FACE OR OF THE SKULL OR OTHER DEFECTS IN THE BODY AREA

(71) Applicant: Horst E. UMSTADT, Weinheim (DE)

(72) Inventor: Horst E. UMSTADT, Weinheim (DE)

(21) Appl. No.: 16/303,822

(22) PCT Filed: May 23, 2017
(86) PCT No.: PCT/EP2017/062376

§ 371 Date: Nov. 21, 2018

(30) Foreign Application Priority Data
May 23, 2016 (DE) 10 2016 109 456.0

(51) Int. Cl. A61F 2/30; A61F 2/28

(52) U.S. Cl. CPC A61F 2/30942; A61F 2/3099; A61F 2/2875; A61F 2002/30952; A61F 2240/004; A61F 2002/30968; A61F 2310/00203; A61F 2310/00239; A61F 2002/30957

(57) ABSTRACT

Method for the production of a temporomandibular joint endoprosthesis, skull prosthesis or body part prosthesis, in which, as first step, an image of the prosthesis to be produced is created, subsequently by a computerized machining process, a size-adapted prosthetic blank is made from a ceramic blank, using said image and in a downstream thermal treatment the prosthetic blank is transformed into the intended temporomandibular joint endoprosthesis, skull prosthesis or body part prosthesis and temporomandibular joint endoprosthesis.

Method for the production of a temporomandibular joint endoprosthesis or implant for the treatment of osseous (bony) deficiencies or defects of the facial bones and the cranium, as well as other osseous defects in the body

The invention concerns a method for the production of a temporomandibular joint endoprosthesis, a skull implant or body part implant as replacement for the original osseous structures.

It is a commonly-known treatment modality, in defects or dysfunctions of the temporomandibular joint or other, smaller joints within the human body, to replace the affected joint, partially or totally, with a prosthesis. Such defects or dysfunctions may be caused by inflammation and/or trauma and/or tumors and/or genetic predisposition. Prosthetic joint replacement, in this context, is also a known treatment modality for the temporomandibular joint.

The natural temporomandibular joint allows rotational as well as horizontal movements of the joint head (condyle), such movements being called translational movements. The translational movements during mouth opening are executed by the lateral pterygoid muscle which is attached to the condyle. That function of the lateral pterygoid muscle is lost in total temporomandibular joint prostheses in which the temporomandibular joint endoprostheses are designed like a ball-and-socket-joint, with only three degrees of freedom. As a consequence, the possibility of translational temporomandibular joint movements during mouth opening is lost as well.

After implantation of a prosthesis based on the typical ball-and-socket-joint, the lower jaw, on that side, merely rotates around a fixed point, so that translational movements of the lower jaw are no longer possible. Such translational movements, however, are necessary for normal mastication. That type of temporomandibular joint endoprostheses is, therefore, uncomfortable for the patient and impedes normal jaw function. It may also even cause damage to the joint socket.

A temporomandibular joint endoprosthesis is known from EP 0 628 293 B1 which incorporates a part for the lower jaw that rotates around a pivot point below the center of the natural joint head, in relation to the skull-side part supporting the socket of the prosthesis. It remains unclear, whether there is a horizontal component in the movement of such a prosthesis and, if so, how great the forces involved are and which muscle/muscles effect such horizontal movement.

This is supposed to allow natural temporomandibular joint movement.

Up to this day, temporomandibular joint prostheses are built from various different materials. For instance, sliding elements are supposed to consist of a polymer, the condyle from aluminum oxide (Al₂O₃), and the part of the skull that supports the socket should be made from a cobalt-chromium-molybdenum alloy. Screws and plates are preferably made from a titanium alloy. Consequently, such temporomandibular joint prostheses present a very complex design and consist of a variety of different materials.

Furthermore, there are types of temporomandibular joint prostheses published which consist, primarily or entirely, of titanium alloys.

The known temporomandibular joint prostheses are either already completely preproduced produced, or they are available at least as semi-finished products. Therefore, it has been necessary up to now to adjust the temporomandibular joint to the temporomandibular joint endoprosthesis to be implanted. It is a disadvantage, here, that—under certain circumstances—substance must be removed from the temporomandibular joint that would be highly worth preserving. This necessity arises from the need to fit the preproduced prosthesis into the remainders of the temporomandibular joint. The attachment of the lateral pterygoid muscle, in particular, often requires resection in this type of temporomandibular joint prostheses, causing an extensive loss of function.

Titanium alloys are known to allow very good integration into the surrounding bone (osseointegration). This is, however, not fully the case where one considers integration of or bonding with soft tissues. It is due to that shortcoming that prostheses from titanium alloys cannot serve as anchorage for soft tissues such as tendons or muscles.

The invention has the object of providing a temporomandibular joint endoprosthesis, skull prosthesis or body part prosthesis that will require the resection of damaged substance from, e.g., the temporomandibular joint only and which will allow a high degree of functionality after healing is accomplished.

The object is achieved by the features of claims 1 and 8. Advantageous embodiments are described in the subclaims.

The first step in the production process of a temporomandibular joint endoprosthesis, skull prosthesis or body part prosthesis, according to the invention, involves making a model of the prosthesis to be produced. Subsequently, a size-adapted ceramic blank is produced in a computerized machining process, using the model prosthesis as template. Downstream thermal treatment yields the temporomandibular joint endoprosthesis, skull prosthesis or body part prosthesis from the ceramic blank.

Where necessary, a model of the bony skull will be obtained first. From that model, using the image data underlying it, the substance to be resected is determined and, then, the plastic template for the endoprosthesis is made. The plastic template constitutes a positive mould, from which—via a 3D-scanning process—a digitized model may be created to fabricate the actual endoprosthesis. It is advantageous, here, that the digital image of the endoprosthesis can initially be used to simulate the planned implantation surgery. Simulation can be performed directly on the model template or on the digital computer image. When performing a computerized simulation, the image data from the skull and the scanned template can be used and blended together. After simulation, the template can be modified, should the simulation have yielded an unsatisfactory result. If the result of the simulation is satisfactory, the template can be used to fabricate the blank for the prosthesis.

One may also imagine an entirely computerized process in which the template exists as computer model only. Modelling and simulation are entirely performed on the computer. That process, then, will not require production of a plastic model.

A particular advantage is gained from the fact that the temporomandibular joint endoprosthesis, skull prosthesis or body part prosthesis is produced after a positive template. Consequently, the individual temporomandibular joint situation dictates the form of the prosthesis. Here, the particular advantage is that only damaged or pathological substance needs to be removed surgically from the temporomandibular joint. It is not necessary to adapt the temporomandibular joint to a preproduced temporomandibular joint endoprosthesis and, in order to achieve that, to remove healthy tissue that would merit being left in place. Each temporomandibular joint endoprosthesis, skull prosthesis or body part prosthesis produced according to this invention is a one-of-a-kind specimen and adapted perfectly to the situation in the individual temporomandibular joint. The fact that the lateral pterygoid muscle can be preserved is a particular advantage, while the rest of the destroyed joint head is replaced. Thereby, the surgeon can preserve the attached muscles during surgery.

The temporomandibular joint endoprosthesis, skull prosthesis or body part prosthesis produced according to this invention integrates well into the surrounding soft tissues and forms a stable connection with the supporting bone. Completion of the healing process guarantees high functionality of the temporomandibular joint replaced with the temporomandibular joint endoprosthesis. Another advantage is presented by the preserved attachments of the muscles of mastication, which, then, preserves the natural rotational and sliding movements.

The process according to this invention is also well-suited to be used in the production of facial bone prostheses, especially in the region of the zygomatic bone, the bony forehead, the chin and the mandibular angle. Another area of application is the production of body part prostheses, for instance, in the area of the iliac crest. All in all, the process according to this invention is particularly advantageous in the production of prostheses for plastic surgery.

The temporomandibular joint endoprosthesis, skull prosthesis or body part prosthesis, according to this invention, consists exclusively of a ceramic material. Ceramic material presents the particular advantage of providing, on the one hand, osseointegration and, on the other hand and as an added feature, soft tissue integration, the latter of which surrounds the temporomandibular joint or the facial bones. Integration into the soft tissues, after healing is completed, allows high functionality of the temporomandibular joint. The temporomandibular joint endoprostheses, skull prostheses or body part prostheses join firmly with their bony support (osseointegration) and integrate into the soft tissues without causing irritation. An additional advantage is that, by virtue of individual production of temporomandibular joint endoprostheses, skull prostheses or body part prostheses, it is possible to achieve an aesthetic temporomandibular joint situation that is very close to the original situation.

The ceramic blank consists of a ceramic powder which, with the help of an organic binder, for instance polyacrylic acid, is compressed to a block. Such a ceramic blank, also called ceramic powder pellet or compact, displays high porosity and little inherent strength. The texture of the ceramic compact resembles that of chalk. This makes it possible to treat the ceramic blank with machining tools. The lack of inherent strength of the ceramic blank allows short machining time and little tool wear.

Downstream thermal treatment compacts the ceramic material, and the organic binder is removed from the matrix of the ceramic material. This causes the prosthetic blank to shrink due to reduced porosity and altered texture. This leads to a loss of volume. Consequently, it is necessary to fabricate a size-adapted prosthetic blank from the ceramic blank. Completion of thermal treatment yields a ceramic temporomandibular joint endoprosthesis, skull prosthesis or body part prosthesis from the prosthetic blank. Such a temporomandibular joint endoprosthesis, skull prosthesis or body part prosthesis is characterized by a high degree of hardness and strength. The bending or flexural strength of zirconium dioxide, for instance, is higher than that of the titanium alloys usually used for prostheses.

Preferably, the ceramic blank is made from oxide ceramics. Oxide ceramics are monophasic ceramic materials without a glassy phase. Although oxide-yielding metals belong to the base metals, they do display high oxidation potential and form very stable oxides.

A preferred design includes ceramic blanks made from zirconium dioxide (ZrO₂). Zirconium dioxide is non-magnetic and very resistant against acids and alkaline lyes as well as other chemical, thermic and mechanical influences. To make the ceramic blank more stable, the sintering powder often contains stabilizing additives in the form of other metal oxides. The use of zirconium dioxide in medicine is already established. By virtue of its high biocompatibility, zirconium dioxide is already in use for the production of hip prostheses (femoral head implants). It is also established to fabricate metal-free dental implants or dentures from zirconium dioxide. When it comes to implants for joint heads, however, the surgeon usually opts for preproduced components. Alternatively, one may also imagine the ceramic blank to be made from aluminum oxide (Al₂O₃). Like zirconium dioxide, aluminum oxide is biocompatible and displays high strength after downstream thermal treatment. The bending or flexural strength, however, is smaller than that of zirconium dioxide.

Preferably, the ceramic blank is subject to upstream thermal treatment. Ceramic sintering powder yields the ceramic blank. Compacting the ceramic sintering powder into a block, using organic binder only, will yield a ceramic blank of a soft, chalk-like consistency. The latter may be easily machinable but excessively difficult to handle. The softness of the texture holds the risk of the machinated ceramic blank being destroyed before it can be subject to downstream thermal treatment. Upstream thermal treatment pre-strengthens the ceramic blank. This will lengthen milling time and increase tool wear, but it will also make the prosthetic blank produced from the ceramic blank more stable and easier to handle. This prevents destruction of the ceramic blank before downstream thermal treatment, which may otherwise happen without upstream thermal treatment, if the ceramic blank is handled wrongly.

Completion of resective surgery, during which the medically necessary and destroyed material was removed from the temporomandibular joint, is ideally followed by digital scanning of the temporomandibular joint area. Then, a CAD (Computer-Aided Design) process will yield an image of the temporomandibular joint endoprosthesis to be produced, using the scanned image data. The process takes into account the shrink during downstream thermal treatment. This is, so that a size-adapted prosthetic blank can be obtained, from which—via downstream thermal treatment—the final, size-compatible, dimension-true temporomandibular joint endoprosthetic may be produced.

Alternatively, one may imagine a model of the temporomandibular joint endoprosthesis, skull prosthesis or body part prosthesis one wants to fabricate to be based on an already existing set of diagnostic findings, for instance on imaging data obtained from Digital Volume Tomography (DVT). The model is preferably made from plastic material. This is advantageous, because it enables the surgeon to test position and form of the temporomandibular joint endoprosthesis, skull prosthesis or body part prosthesis prior to surgery and in the site where surgery will be performed. This allows changes/alterations to be made on the model, meaning to say that the resection is performed on the model and can therefore be viewed as a simulation of the intended surgery. The—possibly modified—model, then, serves as template for the machining production process.

The digital model of the temporomandibular joint endoprosthesis, skull prosthesis or body part prosthesis is subsequently transferred to a computer-controlled milling machine. The machining tool that should preferably be used is a three-dimensional CNC (Computerized Numerical Control) milling machine. CN-controlled machining machines allow computerized numerical control of machining tools and, thereby, high-precision production of prosthetic blanks. The integration of digitized scan image data and digitized modelling of that image data by a CAD process yields a production process that is entirely digitized, meaning to say that the production process of the prosthetic blank is entirely digitized. This Computer-Aided Manufacturing (CAD) process is a cost-efficient as well as high-precision way to fabricate complex prosthetic blanks. This makes it possible to fabricate temporomandibular joint to their individual purpose.

Preferably, the downstream thermal treatment is a sintering process. The sintering process requires the prosthetic blank to be subject to temperatures above 1,000° C., e.g. a temperature of 1,400° C., for several hours, e.g. for eight hours. The prosthetic blank will shrink by 25 Vol % and more under these conditions. Shrink is caused by loss of porosity and changes in the crystalline texture of the ceramic material. The prosthetic blank becomes a ceramic temporomandibular joint endoprosthesis, skull prosthesis or body part prosthesis after thermal treatment. These are characterized by high material strength, excellent biocompatibility and good osseointegration and integration into the adjacent soft tissues.

Ceramic temporomandibular joint endoprostheses, skull prostheses or body part prostheses produced according to this invention offer the advantage of good osseointegration and, furthermore, the advantage of good soft tissue integration. In addition, the ceramic endoprostheses, skull prostheses or body part prostheses provide high material strength, above all flexural strength. This is especially important, because, during mastication, the area of the temporomandibular joints is subject to high mechanical loads. After healing is complete, the good integration of the ceramic temporomandibular joint endoprosthesis, skull prosthesis or body part prosthesis into both the bony tissue and, simultaneously, the soft tissues, ensures high functionality of the prosthetically treated temporomandibular joint. At the same time, individual modelling of the prosthesis ensures a highly aesthetical outcome, which is especially relevant in cases with facial implants.

An advantageous design involves the temporomandibular joint endoprosthesis, skull prosthesis or body part prosthesis bearing inserts to retain fastening fixtures in the ceramic material. These inserts preferably come in the form of drill holes with countersinks or counterbores in which the outer part may be tapered and have a conical shape. This allows the complete incorporation of screw-like fixtures, for instance titanium screws. The use of such fastening fixtures enables the surgeon to affix the temporomandibular joint endoprosthesis, skull prosthesis or body part prosthesis firmly and stably. Consequently, this facilitates osseointegration of the temporomandibular joint endoprosthesis, skull prosthesis or body part prosthesis. Preferably, fastening fixtures include titanium screws on account of their own, added, good osseointegration. After healing, the endoprosthesis itself is firmly anchored within the bone, also. Alternatively, preproduced screws may be used, which are also made from zirconium dioxide.

The following will give a more detailed explanation of the production process for temporomandibular joint endoprostheses, skull prostheses or body part prostheses according to this invention:

First, a ceramic blank is made. This ceramic blank consists of a powdery ceramic material, for instance aluminum oxide (Al₂O₃), preferably zirconium dioxide (ZrO₂). The powdery material is compacted to a block and kept in the desired shape by adding an organic binder based on polyacrylic acid. Before machinating it with machining tools, the ceramic blank may be subject to upstream thermal treatment. Such upstream thermal treatment is also called preliminary firing. Preliminary firing is associated with less shrink and pre-strengthens the ceramic material. This facilitates handling of the ceramic blank as well as the prosthetic blank produced by machining.

The use of a 3D scanner, followed by CAD, creates a digital model of the prosthesis to be produced. The dimensions of the model take into account the shrink caused by thermal treatment during production. This means to say that the prosthetic blank is size-adapted in relation to the future temporomandibular joint endoprosthesis, skull prosthesis or body part prosthesis.

As an alternative to scanned image data, one may begin by creating a template of the temporomandibular joint endoprosthesis, skull prosthesis or body part prosthesis to be produced from initial findings based on x-rays or similar. This template, for instance, may then be converted to a machine-readable model, including, where necessary, modifications to the original template as a result of digital surgery simulation. The simulation may be performed using a physical template or it may be entirely computer-based using existing data of the skull and a digitized set of data from the physical template. The template is preferably made of plastic material. It may be imagined, here, that the template may also be created using a 3D-plotter. Alternatively, production of the template may also be performed mechanically by workmanship, or one uses a template to start from which exists as computer model exclusively.

The digitally created model of the temporomandibular joint endoprosthesis, skull prosthesis or body part prosthesis to be produced is transferred onto a computer-controlled, three-dimensional milling machine which fabricates the prosthetic blank from a ceramic blank via machining process, using the digital model. Subsequently, the prosthetic blank is subject to downstream thermal treatment, which transforms the prosthetic blank into the temporomandibular joint endoprosthesis, skull prosthesis or body part prosthesis. Upstream thermal treatment involves thermal treatment over a period of eight hours, at a temperature of 1,400° C.

During computerized modelling of the model for the temporomandibular joint endoprosthesis, skull prosthesis or body part prosthesis, inserts for fastening fixtures are added. These are designed in the shape of cylindrical drill holes, while the outer part of the drill hole is designed as tapered countersink or counterbore to accommodate the screw heads. The drill holes serve to accommodate fastening fixtures such as, preferably, titanium screws or, alternatively, screws made from a ceramic material.

1. Method for the production of a temporomandibular joint endoprosthesis, skull prosthesis or body part prosthesis, in which, as first step, an image of the prosthesis to be produced is created, subsequently by a computerized machining process, a size-adapted prosthetic blank is made from a ceramic blank, using said image and in a downstream thermal treatment, the prosthetic blank is transformed into the intended temporomandibular joint endoprosthesis, skull prosthesis or body part prosthesis.

2. Method according to claim 1, characterized in that the ceramic blank comprises zirconium dioxide.

3. Method according to claim 1, characterized in that the ceramic blank comprises aluminum oxide.

4. Method according to any of the claims 1 to 3, characterized in that the ceramic blank was subject to upstream thermal treatment.

5. Method according to any of the claims 1 to 4, characterized in that the creation of a model comprises a combined process of scanning and CAD.

6. Method according to any of the claims 1 to 4, characterized in that a model is produced to create the image.

7. Method according to any of the claims 1 to 6, characterized in that the digital machining process is a CNC processed three-dimensional milling process.

8. Method according to any of the claims 1 to 7, characterized in that the downstream thermal treatment is a sintering process.

9. Temporomandibular joint endoprosthesis, skull prosthesis or body part prosthesis made from ceramic material, produced by a process according to one of the above-mentioned claims.

10. Temporomandibular joint endoprosthesis, skull prosthesis or body part prosthesis according to claim 9, characterized in that recesses for fastening elements are brought into the ceramic material.
